(19) United States
(12) Patent Application Publication
(10) Pub. No.: US 2025/0166824 A1
(43) Pub. Date: May 22, 2025

(54) System and Method for Annotating Pathology Images to Predict Outcome

(71) Applicants: Vinay PULIM, Brooklyn, NY (US); THE JOHNS HOPKINS UNIVERSITY, Baltimore, MD (US)

(72) Inventors: Janis M. Taube, Baltimore, MD (US); Julie Stein, Bethesda, MD (US); Vinay Pulim, Brooklyn, NY (US); Tricia Ruth Cottrell, Kingston (CA)

(21) Appl. No.: 18/841,323

(22) PCT Filed: Feb. 24, 2023
(86) PCT No.: PCT/US2023/013869

§ 371 Date: Aug. 23, 2024

Related U.S. Application Data
(60) Provisional application No. 63/313,548, filed on Feb. 24, 2022.

(51) Int. Cl. G16H 50/20; G06V 10/26; G06V 10/764; G06V 20/69; G06V 20/70; G16H 10/60

(52) U.S. Cl. CPC G16H 50/20; G06V 10/26; G06V 10/764; G06V 20/695; G06V 20/698; G06V 20/70; G16H 10/60; G06V 2201/03

(57) ABSTRACT

Methods, systems, apparatus, and computer programs, for predicting clinical outcome of a patient. In one aspect, a method includes obtaining a pathology image associated with the patient; processing the pathology image including: determining a region of interest of the pathology image; and segmenting the pathology image into a plurality of tiles, wherein the plurality of tiles has a uniform size; providing the processed pathology image as an input to a machine learning model configured to annotate the pathology image and predict the clinical outcome of the patient based on the annotated pathology image; obtaining, as an output of the machine learning model, the annotated pathology image associated with the patient, wherein the annotated pathology image includes annotations for different classes of tissues including a tumor regression; and determining, based on the annotated pathology image, a metric indicative of the clinical outcome of the patient.

### CROSS-REFERENCE TO RELATED APPLICATIONS

This application claims priority to U.S. Provisional Patent Application No. 63/313,548, filed on Feb. 24, 2022, which is incorporated herein by reference in its entirety.

### TECHNICAL FIELD

The present disclosure is directed towards predicting patient outcome, e.g., responsiveness to treatment regimens, based on pathology image analysis.

### SUMMARY

According to one innovative aspect of the present disclosure, a computer-implemented method for predicting clinical outcome of a patient, the method comprising: obtaining a pathology image associated with the patient; processing the pathology image including: determining a region of interest of the pathology image; and segmenting the pathology image into a plurality of tiles, wherein the plurality of tiles has a uniform size; providing the processed pathology image as an input to a machine learning model configured to annotate the pathology image; obtaining, as an output of the machine learning model, the annotated pathology image associated with the patient, wherein the annotated pathology image includes annotations for different classes of tissues including a tumor regression; and determining, based on the annotated pathology image, a metric indicative of the clinical outcome of the patient.

Other versions include corresponding systems and computer programs to perform the actions of methods defined by instructions encoded on computer readable storage devices.

These and other versions may optionally include one or more of the following features. For instance, in some implementations, the clinical outcome of the patient includes responsiveness to one or more treatment regimens, survival, disease recurrence, and disease progression.

In some implementations, the pathology image includes a histopathology slide image.

In some implementations, the machine learning model has been trained on a plurality of training data items. Each training data item includes an annotated pathology image with tissue classes.

In some implementations, generating rendering data, when rendered by a user device, causes the user device to display a user interface that displays the annotated pathology image associated with the patient and the predicted clinical outcome of the patient.

In some implementations, the user interface includes a user selectable element to upload the pathology image for applying the machine learning model.

In some implementations, the different classes of tissues include tumor, necrosis, area of tumor regression, and normal background. The normal background indicates an uninvolved tissue.

In some implementations, the metric indicative of the clinical outcome of the patient includes a percent residual viable tumor. The percent residual viable tumor is computed based on areas of the different classes of tissues in the pathology image.

In some implementations, the metric indicative of the clinical outcome of the patient includes a percent regression and a percent necrosis in the pathology image.

In some implementations, processing the pathology image further includes: normalizing color of the pathology image based on color of a set of pathology images; and applying image transformations to the pathology image. The image transformations include rotation, shift, flip, zoom, affine transformation, and adjusting brightness and contrast.

These and other aspects of the present disclosure are discussed in more detail in the detailed description below with reference to the accompanying drawings.

### BRIEF DESCRIPTION OF THE DRAWINGS

FIG. 1 is a block diagram of an example of a system for predicting patient outcome based on annotated pathology images.

FIG. 2 is a block diagram of an example of a system for training a machine learning model.

FIGS. 3A-3B show performance of a machine learning model.

FIG. 4A shows an example user interface for annotating pathology images and predicting patient outcome.

FIG. 4B shows an example of an annotated pathology image and a predicted patient outcome.

FIG. 5 is a flowchart of an example of a process for applying a machine learning model to predict patient outcome based on annotated pathology images.

FIG. 6 is a flowchart of an example of a process of training a machine learning model to annotate pathology image used in predicting patient outcome.

FIG. 7 is an example of a block diagram of system components that can be used to implement a system for predicting patient outcome based on annotated pathology images.

### DETAILED DESCRIPTION

According to one innovative aspect of the present disclosure, systems, methods, and computer programs for predicting patient outcome(s) based on a pathology image of a cancer from the patient are disclosed. The patient outcome (also referred to as clinical outcome of the patient) can include responsiveness to treatment (including pharmaceutical product, immunotherapy, and chemotherapy), survival, disease recurrence, and/or disease progression, etc. In some implementations, the present disclosure can be used to annotate different tissue classes, e.g., tumor, necrosis, tumor regression, in the pathology image. However, the present disclosure need not be so limited, and instead, can be applied to non-tumor samples, as well as frozen samples. In addition, the present disclosure need not be limited to human samples. Instead, for example, samples analyzed by the present disclosure can include human or animal samples. The pathology image (e.g., image scanned by a slide imaging scanner) can include a histopathology (tissue) image.

The system and methods of the present disclosure solve a particular problem known in the field related to the prediction of patient outcome such as responsiveness to therapeutic regimens, e.g., anti-PD-1-based immunotherapies. Current approaches include radiologic assessments of response to therapy, however, these are insufficient to correctly predict patient outcome. Interpretation and scoring of slides can be laborious, and it is not possible to process data fast enough to provide useful insights in timely manner for a meaningful number of patients. Moreover, these current systems—such as a pathologist visually scoring a slide by looking through a microscope at the slide(s) or a digitized slide(s)—lend themselves to rather subjective predictions amongst interpreters, motivating for a standardized assessment of pathologic response to therapeutic regimens. To solve this problem of developing standardized pathologic response criteria, the present disclosure, computationally annotates different classes of tissues, or cells, in pathology images and efficiently computes a metric predictive of the patient outcome. The metrics, e.g., percent residual viable tumor (also referred to as % RVT), necrosis, and regression can be used to predict the patient outcome including responsiveness to immunotherapy. Regression can refer to the area that used to be occupied by tumor after the tumor has decreased in area. The regression area is no longer tumor, but is also distinguished from ‘normal’ background, uninvolved tissues. The present disclosure also describes a user interface that allows a user to apply the system and view annotated pathology images and predicted patient outcome.

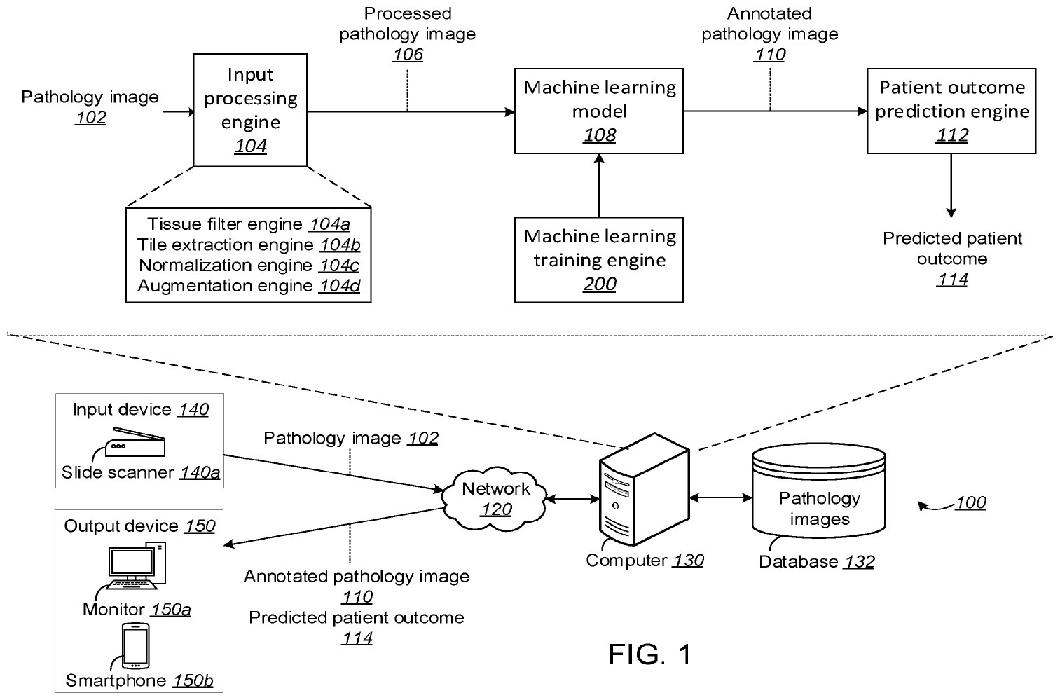

FIG. 1 is a block diagram of an example of a system 100 that predicts patient outcome based on annotated pathology images. The system 100 includes an input device 140, a network 120, and a computer 130. The computer 130 can include an input processing engine 104, a machine learning model 108, and a patient outcome prediction engine 112. In some implementations, the computer 130 can be a server. For purposes of the present disclosure, an “engine” can include one or more software modules, one or more hardware modules, or a combination of one or more software modules and one or more hardware modules.

The input device 140 can include any device that is configured to generate a pathology image 102 of one or more organs of a patient's body, a device that is configured to provide a generated pathology image 102 of one or more organs of a patient's body, or any combination thereof. By way of example, the input device 140 can include a slide scanner 140a that is configured to scan one or more pathology slides (e.g., melanoma resection, tissue biopsy of one or more organs of a patient's body. The pathology slides may be tissue-level slides or cell-level slides. In some implementations, the slide scanner can be a whole slide scanner. In some implementations, the slide scanner 140a can generate the pathology image 102 and transmit the pathology image 102 to another device such as the computer 130 across the network 120. Alternatively, the slide scanner 140a can generate the pathology image 102 that can be accessed by one or more other input devices 140 such as computer (e.g., desktop, laptop, tablet, etc.), a smartphone, or a server. In such instances, the one or more other input devices can access the pathology image 102 generated by the slide scanner 140a and transmit the accessed pathology image to the computer 130 via the network 120.

The network 120 can include one or more of a wired Ethernet network, a wired optical network, a wireless WiFi network, a LAN, a WAN, a Bluetooth network, a cellular network, the Internet, or any combination thereof.

Though examples of the present disclosure described herein refer to the pathology image 102 and the slide scanner 140a, the present disclosure is not so limited. Instead, the present disclosure can include machine learning models (e.g., one or more neural networks) that have been trained to predict patient outcome based on any type of image. Such images can include two-dimensional images, three-dimensional images, image models generated based on another image, or the like.

The computer 130 is configured to obtain one or more pathology images 102 generated by the input device 140 such as the slide scanner 140a. In some implementations, the one or more pathology images can be an image received over the network 120. In other implementations, the computer 130 can access pathology images stored in a database 132 that is accessible to the computer 130. The database 132, a local database or a cloud-based database, can include pathology images, patient identifiers, patient medical history including current and past treatment regimens, and predicted patient outcome. The pathology images in the database can be raw or annotated pathology images. In some implementations, the patient identifiers may be encrypted. However, the present disclosure is not limited to use of encrypted patient identifiers.

The computer 130 can provide an obtained pathology image such as the pathology image 102 to an input processing engine 104. In some implementations, the computer 130 can provide the obtained pathology image for input to the input processing engine 104 in the same format the pathology image 102 was in when the pathology image 102 was received. For example, the computer 130 can receive a two-dimensional image and provide the two-dimensional image as an input to the input processing engine 104. In some implementations, the computer 130 can generate a three-dimensional pathology image from the two-dimensional pathology image and provide the three-dimensional pathology image for input to the input processing engine 104.

The input processing engine 104 is configured to receive the pathology image 102 and generate progressed pathology image 106 for input to the machine learning model 108. In some implementations, the pathology image 102 is an image with about 50k by 50k pixels. Referring to FIG. 1, the input processing engine 104 can perform one or more steps of tissue filtering (e.g., using a tissue filter engine 104a), tile extraction (e.g., using a tile extraction engine 104b), normalization (e.g., using a normalization engine 104c), and augmentation (e.g., using an augmentation engine 104d). For example, the tissue filter engine 104a computes tissue mask(s) and extract portions of the pathology image 102 that constitute as tissues. The tile extraction engine 104b segments the pathology image 102, either before or after the tissue filtering, into a set of tiles. In some implementations, each tile is an image with 96 by 96 pixels. The tile extraction engine 104b can determine an optimal size of the tile. The normalization engine 104c normalizes colors of the pathology image 102, e.g., based on mean and distribution of the color across multiple pathology images. The augmentation engine 104d applies image transformations (also referred to as noise or noises), e.g., rotation, shift, flip, zoom, affine transformation, adjusting brightness and/or contrast, to the pathology image 102. This image augmentation prevents the machine learning model 108 from overfitting. The processed pathology image 106 generated by the input processing engine 104 can include a data structure that represents the pathology image. The processed pathology image 106 outputted by the input processing engine 104 can be provided as input to the machine learning model 108. In the example of FIG. 1, the processed pathology image 106 is a data structure that represents the (e.g., two-dimensional) pathology image 102 that is processed through the engines 104a-104d.

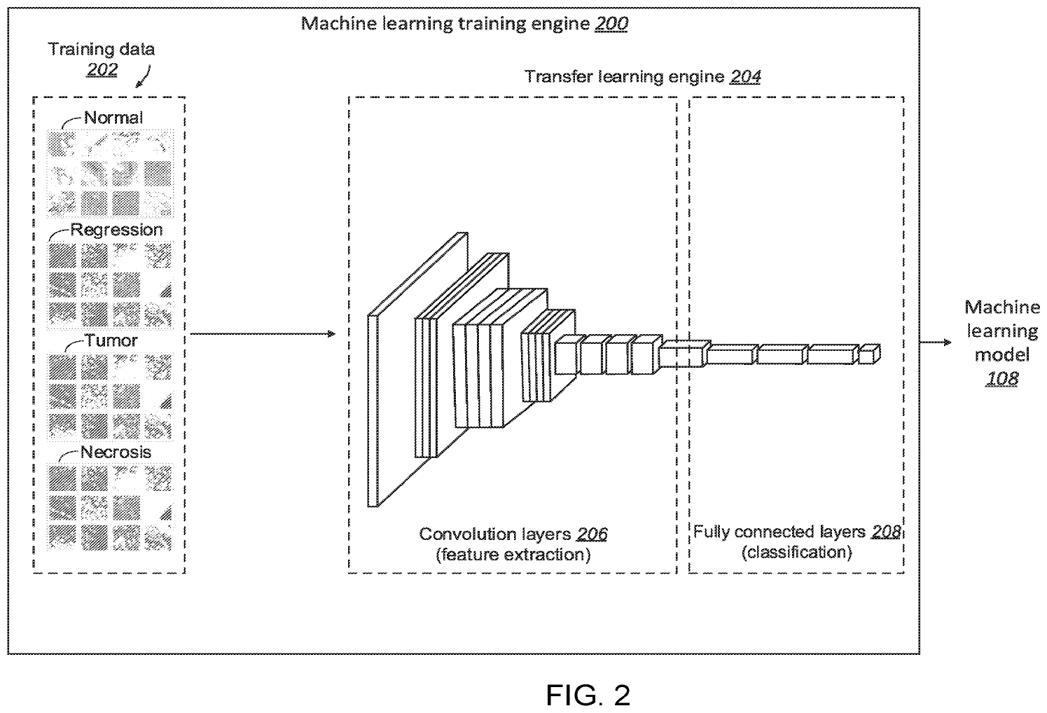

The machine learning model 108 is configured to receive the processed pathology image 106 and generates annotated pathology images 110. The machine learning model 108 is a model outputted from training a machine, e.g., the computer 130, to annotate the processed pathology image 106; training is described more in details referring to FIG. 2 below. The annotated pathology image 110 includes annotations for each tissue class. For example, particular areas of the pathology image are annotated as (predicted) tumor. Similarly, particular areas of the pathology image are annotated as necrosis. In some implementations, the annotations are graphical annotations, e.g., boundaries and/or areas. In some implementations, each area (e.g., uniform sized tile) can be assigned a single tissue class; for example, a particular tile cannot be annotated as both normal and tumor. The annotation is based on class membership probabilities predicted by the machine learning model 108. For example, for a particular tile predicted to be 70% tumor, 10% regression, 20% normal, the particular tile is annotated with a label tumor. In some implementations, each tile can have multiple labels based on a probability threshold for each membership; that is, a particular tile predicted to be 40% tumor, 40% regression, 20% normal (e.g., using a probability threshold of 33%) is annotated with both tumor and regression. The annotated pathology image outputted from the machine learning model 108 can be provided as an input to the patient outcome prediction engine 112. In some implementations, a summary report is provided as an output of the machine learning model 108, where the summary report includes respective areas for each tissue class. The summary report can be provided in addition to or instead of the annotated pathology image. In some implementations, predicting patient outcome is also performed by the machine learning model 108.

The patient outcome prediction engine 112 is configured to receive the annotated pathology image 110, the summary report, or both, and generates predicted patient outcome 114. The patient outcome (also referred to as clinical outcome) includes responsiveness to treatment regimens including pharmaceutical product, immunotherapy, and chemotherapy, patient survival, disease recurrence, and disease progression. In some implementations, the patient outcome prediction engine 112, determines a metric indicative of the clinical outcome of the patient. For example, in some implementations, the metric can be a percent residual viable tumor (RVT), where the percent residual viable tumor can be computed based on respective areas of the different tissue classes.

In some implementations, for example, the percent RVT can be calculated using the following expression:

$$\% RVT = \frac{{Area}_{tumor}}{{Area}_{tumor} + {Area}_{necrosis} + {Area}_{regression}} ,$$

where areas represent sizes in pixels². For example, for a pathology image with 562 pixels², 2,612 pixels², 3,286 pixels², the patient outcome prediction engine 112 computes % RVT as 9%. The patient outcome prediction engine 112, based on the computed metric such as % RVT, determines a clinical outcome of the patient. In some implementations, a threshold on the metric is determined by the patient outcome prediction engine 112. For example, less than 10% RVT represents being highly responsive to immunotherapy. In some implementations, a threshold on the metric is determined by medical experts. In some implementations, the machine learning model 108 determines an optimal threshold based on patient outcomes of a set of patients, e.g., patients who undertook the same type of immunotherapy. In some implementations, a metric indicative of the clinical outcome of the patient is a percent regression and a percent necrosis in the pathology image.

In some implementations, the computer 130 can generate rendering data that, when rendered by a device having a display such as an output device 150, can cause the device to output data including annotated pathology image 110 and predicted patient outcome 114. Such rendering data can be transmitted, by the computer 130, to the output device 150 through the network 120 and processed by the output device 150 such as a monitor 150a or a smartphone 150b to generate output data. In some implementations, the output device 150 can be coupled to the computer 130. In such instances, the rendered data can be processed by the computer 130, and cause the computer 130 to output data that include the annotated pathology image 110 and the predicted patient outcome 114.

FIG. 2 is a block diagram of an example of a system, machine learning training engine 200, for training the machine learning model 108. For example, the machine learning model can be trained to distinguish between tumor and non-tumor in the pathology image. Such training can be achieved by obtaining training data 202 and processing the training data 202. The training data 202 includes one or more pathology images for each tissue, or cell, class-such as normal, regression, tumor, and necrosis. However, the present disclosure need not be so limited to these tissue types, and instead, can be applied to any tissues or cell types. In some implementations, the training data 202 are processed using the input processing engine 104 as described referring to FIG. 1. One or more steps of tissue filtering, tile extraction, normalization, and augmentation (described above) can be applied to the training data 202.

In some implementations, the machine learning training engine 200 includes a transfer learning engine 204. The transfer learning engine 204 processes the obtained training data 202 that are labeled with different tissue classes. The transfer learning engine 204 can include convolution layers 206 and fully connected layers 208. The transfer learning engine 204 trains the machine learning model 108 by performing the following steps: (i) freeze convolutional layers, (ii) learn weights for fully connected layers, (iii) unfreeze convolution layers, and (iv) learn weights for all layers, where weights represent learnable parameters of the machine learning model 108. In some implementations, the transfer learning engine 204 uses DenseNet169 architecture including the following layers: convolution+rectified linear unit (ReLU), max pooling (e.g., for downsampling), fully connected+ReLU, and softmax (e.g., that transforms arbitrary real values into probabilities). The convolution layers 206 includes the convolution+ReLU and max pooling layers. The fully connected layers 208 includes the fully connected+ReLU and softmax layers. After learning weights through the transfer learning engine 204, the machine learning training engine 200 outputs the machine learning model 108. In some implementations, the trained machine learning model 108 can be saved in the database 132.

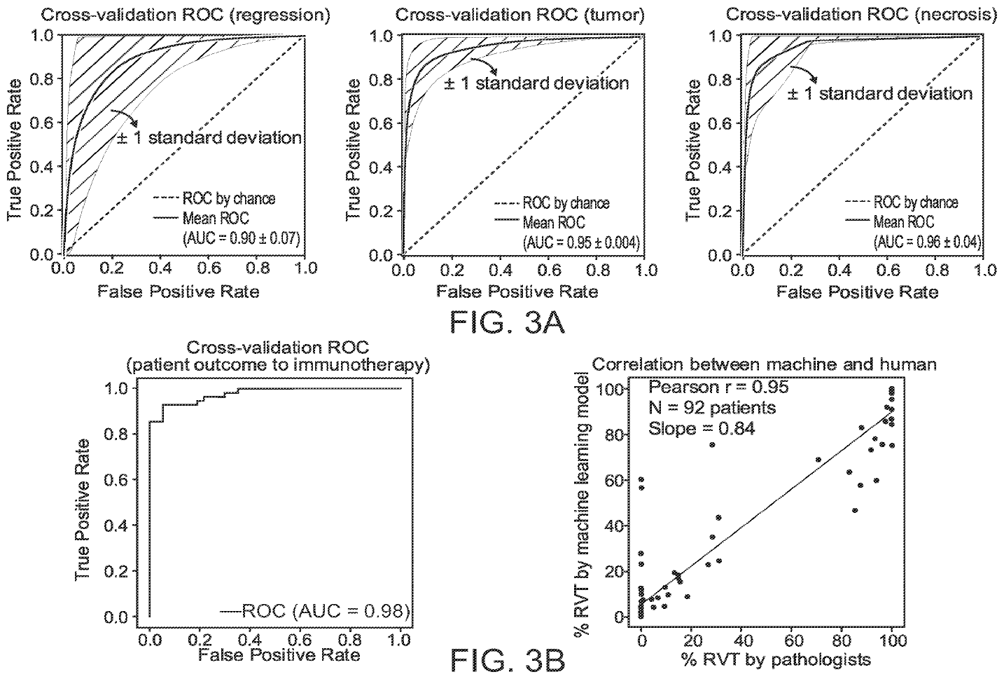

FIGS. 3A-3B show performance of the machine learning model 108 in annotating pathology images. Referring to FIG. 3A, in some implementations, the machine learning training engine 200 can test the machine learning model 108 against testing data. The testing data can be held-out validation data during training or independent testing data. For example, FIG. 3A shows receiver operating characteristic (ROC) curves for correctly predicting different tissue classes, such as regression, tumor, and necrosis, when compared against expert annotation. In some implementations, predicting different tissue classes are performed for each tile in a set of tiles in the pathology image. In some implementations, the expert annotation represents annotations by pathologists—that is, pathologist visual scoring—for each (digitized) slide. As described in FIG. 3A, the machine learning model 108 has superior performance significantly more than by chance.

FIG. 3B shows performance of the machine learning model in predicting patient outcome. In some implementations, predicting patient outcome is based on the annotated pathology image, e.g., respective areas of different tissue classes. In some implementations, portions of the pathology image are used for predicting patient outcome. Referring to FIG. 3B, an area under the ROC curve in correctly predicting the patient outcome to immunotherapy is 0.98. Here, the patient outcome is based on the % RVT as described above. The correlation between the prediction by the machine learning model 108 and the expert across 92 patients is shown to be high with Pearson correlation coefficient (r) of 0.95, with a fitted line with a slope of 0.84.

Based on testing the performance of the machine learning model 108, the machine learning training engine 200 can determine that the machine learning model 108 meets a predefined threshold, e.g., on accuracy and computational speed. Upon determining that the machine learning model 108 does not meet the predefined threshold, the machine learning training engine 200 can adjust training parameters such as a machine learning architecture as shown in FIG. 2, training data 202 (e.g., by obtaining more training data and/or by verifying labels), and retrain the machine learning model 108 based on the adjusted training parameters.

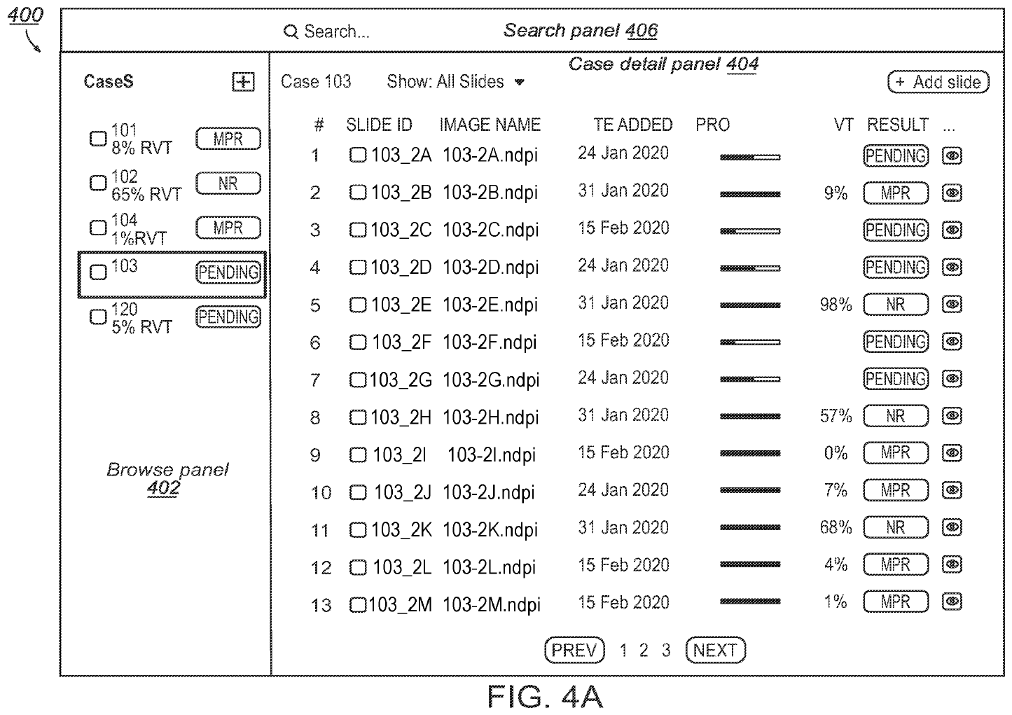

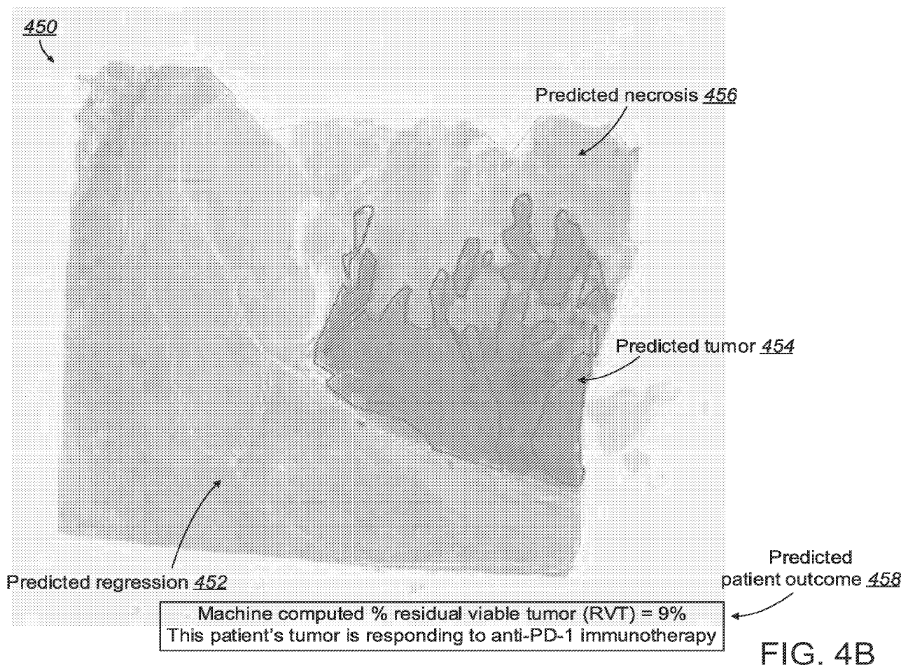

FIG. 4A shows an example user interface 400 for annotating pathology images and predicting patient outcome. The user interface 400 is displayed on the output device 150 such as the monitor 150a or smartphone 150b. The user interface 400, in some implementations, can include a browse panel 402 and a case detail panel 404. The browse panel 402 allows the user to browse through different cases, e.g., cases belonging to a particular disease or a particular patient. Responsive to a selection of the case from the browse panel 402, the case detail panel 404 displays pathology images. For example, each row shown in FIG. 4A represents each pathology image of the case 103. In some implementations, the case detail panel 404 can display pathology image identifier (“SLIDE ID”), image name, date added, progress (e.g., whether the uploaded pathology image is annotated), and predicted patient outcome (e.g., “% RVT”). In some implementations, the user can see—a final result category such as “major pathologic response” or “pathologic complete response” or other associated terminology associated with medically-meaningful categorizations by elements detected by this system, either at the slide-level or case-level. Additionally, incomplete jobs can be seen. In some implementations, the user interface 400 includes a search panel 406 that takes a user input for a particular pathology image (e.g., slide identifier) and presents filtered results that meet the user input.

FIG. 4B shows an example of an annotated pathology image 450 and a predicted patient outcome 458. The annotated pathology image 450 is displayed on the user interface 400 in respond to the user's selection of an image associated with a patient. In some implementations, the annotated pathology image 450 can include different classes of tissues (or cells). For example, predicted regression (areas) 452, predicted tumor (areas) 454, and predicted necrosis (areas) 456 represent areas distinct by tissue classes in the annotated pathology image 450. The user interface 400 can display different tissue classes by boundaries and/or different colors. In some implementations, the annotated pathology image 450 includes information indicative of the predicted patient outcome 458, e.g., a dashboard or pop-up window that summarizes predicted patient outcome (e.g., “This patient's tumor is responding to anti-PD-1 immunotherapy”). In some implementations, the dashboard includes a metric used to predict patient outcome. For example, “machine computed % residual viable tumor (RVT)=9%.” The predicted patient outcome 458 is based on a threshold on the metric determined by experts and/or the machine learning model 108, e.g., less than 10% RVT for being responsive to immunotherapy.

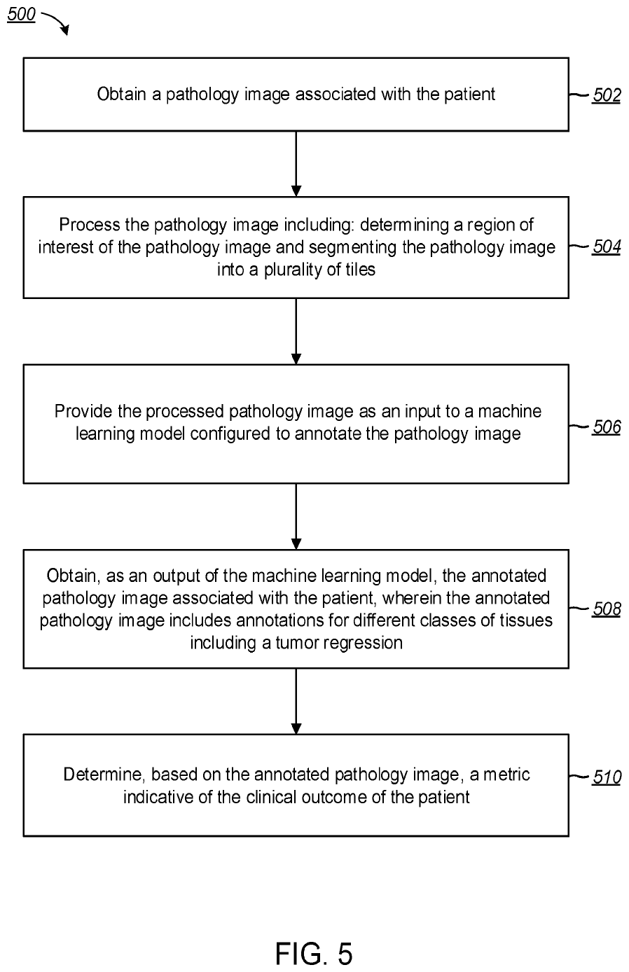

FIG. 5 is a flowchart of an example of a process 500 for applying a machine learning model to predict clinical outcome of a patient. In general, the process 500 includes obtaining a pathology image associated with the patient (502); processing the pathology image including: determining a region of interest of the pathology image; and segmenting the pathology image into a plurality of tiles (504); providing the processed pathology image as an input to a machine learning model configured to annotate the pathology image (506); obtaining, as an output of the machine learning model, the annotated pathology image associated with the patient, wherein the annotated pathology image includes annotations for different classes of tissues including a tumor regression (508); and determining, based on the annotated pathology image, a metric indicative of the clinical outcome of the patient (510). Thus, the process 500 enables a machine learning model that facilitates a prediction of the clinical outcome of the patient based on the annotated pathology image generated by the machine learning model.

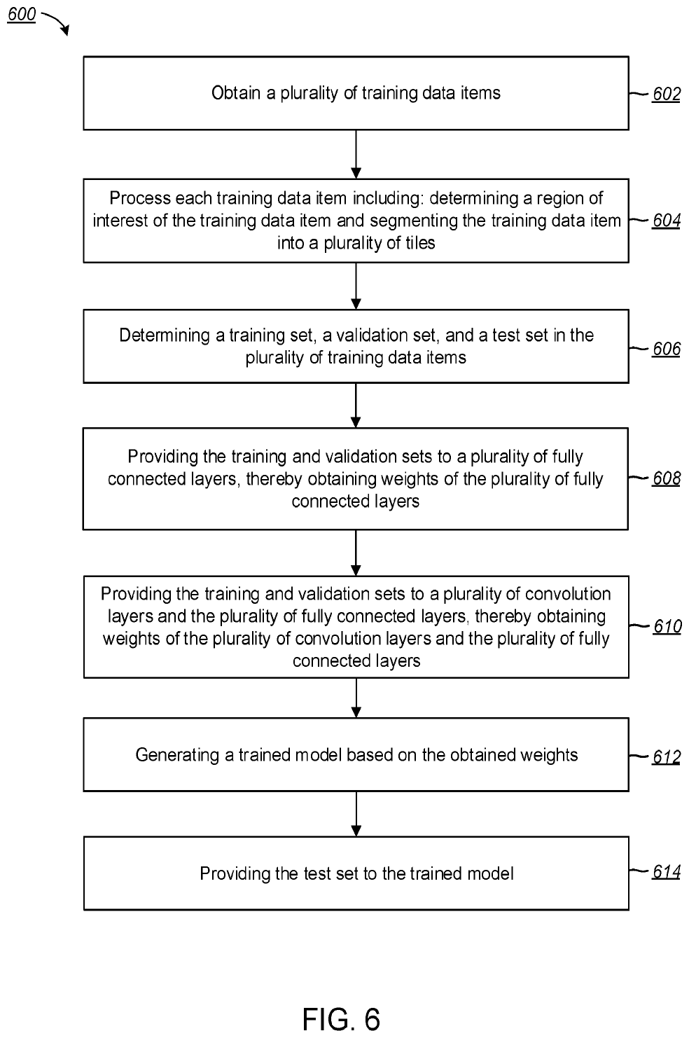

FIG. 6 is a flowchart of an example of another process 600 for training the machine learning model to annotate pathology image used in predicting clinical outcome of a patient. In general, the process 600 includes obtaining a plurality of training data items (e.g., each training data item includes a pathology image that is annotated with tissue classes such as normal, regression, tumor, and necrosis) (602); process each training data item including: determining a region of interest of the training data item and segmenting the training data item into a plurality of tiles (604); determining a training set, a validation set, and a test set in the plurality of training data items (606); providing the training and validation sets to a plurality of fully connected layers, thereby obtaining weights of the plurality of fully connected layers (608); providing the training and validation sets to a plurality of convolution layers and the plurality of fully connected layers, thereby obtaining weights of the plurality of convolution layers and the plurality of fully connected layers (610); generating a trained model based on the obtained weights (612), and providing the test set to the trained model (614).

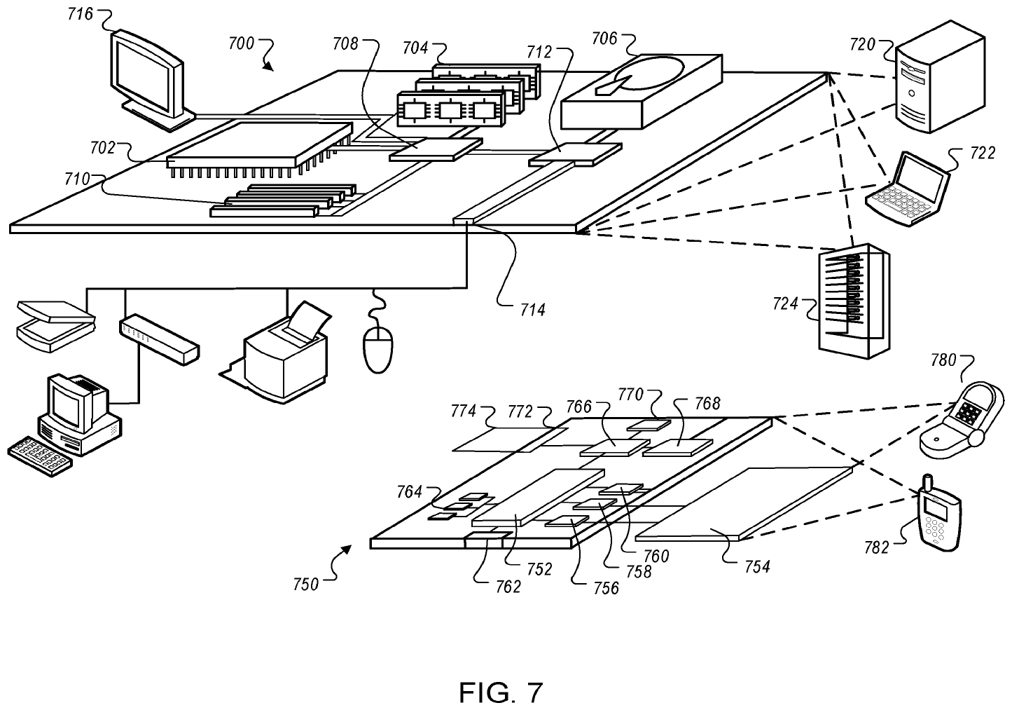

FIG. 7 is an example of a block diagram of system components that can be used to implement a system for predicting patient outcome based on annotated pathology images.

Computing device 700 is intended to represent various forms of digital computers, such as laptops, desktops, workstations, personal digital assistants, servers, blade servers, mainframes, and other appropriate computers. Computing device 750 is intended to represent various forms of mobile devices, such as personal digital assistants, cellular telephones, smartphones, and other similar computing devices. Additionally, computing device 700 or 750 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 700 includes a processor 702, memory 704, a storage device 706, a high speed controller 708 connecting to memory 704 and high-speed expansion ports 710, and a low speed controller 712 connecting to low speed bus 714 and storage device 706. Each of the components 702, 704, 706, 708, 710, and 712, are interconnected using various busses, and can be mounted on a common motherboard or in other manners as appropriate. The processor 702 can process instructions for execution within the computing device 700, including instructions stored in the memory 704 or on the storage device 706 to display graphical information for a GUI on an external input/output device, such as display 716 coupled to high speed controller 708. In other implementations, multiple processors and/or multiple buses can be used, as appropriate, along with multiple memories and types of memory. Also, multiple computing devices 700 can be connected, with each device providing portions of the necessary operations, e.g., as a server bank, a group of blade servers, or a multi-processor system.

The memory 704 stores information within the computing device 700. In one implementation, the memory 704 is a volatile memory unit or units. In another implementation, the memory 704 is a non-volatile memory unit or units. The memory 704 can also be another form of computer-readable medium, such as a magnetic or optical disk.

The storage device 706 is capable of providing mass storage for the computing device 700. In one implementation, the storage device 706 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 704, the storage device 706, or memory on processor 702.

The high speed controller 708 manages bandwidth-intensive operations for the computing device 700, while the low speed controller 712 manages lower bandwidth intensive operations. Such allocation of functions is exemplary only. In one implementation, the high speed controller 708 is coupled to memory 704, display 716, e.g., through a graphics processor or accelerator, and to high-speed expansion ports 710, which can accept various expansion cards (not shown). In the implementation, low speed controller 712 is coupled to storage device 706 and low speed bus 714. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 700 can be implemented in a number of different forms, as shown in the figure. For example, it can be implemented as a standard server 720, or multiple times in a group of such servers. It can also be implemented as part of a rack server system 724. In addition, it can be implemented in a personal computer such as a laptop computer 722. Alternatively, components from computing device 700 can be combined with other components in a mobile device (not shown), such as device 750. Each of such devices can contain one or more of computing device 700, 750, and an entire system can be made up of multiple computing devices 700, 750 communicating with each other.

The computing device 700 can be implemented in a number of different forms, as shown in the figure. For example, it can be implemented as a standard server 720, or multiple times in a group of such servers. It can also be implemented as part of a rack server system 724. In addition, it can be implemented in a personal computer such as a laptop computer 722. Alternatively, components from computing device 700 can be combined with other components in a mobile device (not shown), such as device 750. Each of such devices can contain one or more of computing device 700, 750, and an entire system can be made up of multiple computing devices 700, 750 communicating with each other.

Computing device 750 includes a processor 752, memory 764, and an input/output device such as a display 754, a communication interface 766, and a transceiver 768, among other components. The device 750 can also be provided with a storage device, such as a micro-drive or other device, to provide additional storage. Each of the components 750, 752, 764, 754, 766, and 768, are interconnected using various buses, and several of the components can be mounted on a common motherboard or in other manners as appropriate.

The processor 752 can execute instructions within the computing device 750, including instructions stored in the memory 764. The processor can be implemented as a chipset of chips that include separate and multiple analog and digital processors. Additionally, the processor can be implemented using any of a number of architectures. For example, the processor can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 750, such as control of user interfaces, applications run by device 750, and wireless communication by device 750.

Processor 752 can communicate with a user through control interface 758 and display interface 756 coupled to a display 754. The display 754 can be, for example, a TFT (Thin-Film-Transistor Liquid Crystal Display) display or an OLED (Organic Light Emitting Diode) display, or other appropriate display technology. The display interface 756 can include appropriate circuitry for driving the display 754 to present graphical and other information to a user. The control interface 758 can receive commands from a user and convert them for submission to the processor 752. In addition, an external interface 762 can be provide in communication with processor 752, so as to enable near area communication of device 750 with other devices. External interface 762 can provide, for example, for wired communication in some implementations, or for wireless communication in other implementations, and multiple interfaces can also be used.

The memory 764 stores information within the computing device 750. The memory 764 can be implemented as one or more of a computer-readable medium or media, a volatile memory unit or units, or a non-volatile memory unit or units. Expansion memory 774 can also be provided and connected to device 750 through expansion interface 772, which can include, for example, a SIMM (Single In Line Memory Module) card interface. The expansion memory 774 can provide extra storage space for device 750, or can also store applications or other information for device 750. Specifically, expansion memory 774 can include instructions to carry out or supplement the processes described above, and can include secure information also. Thus, for example, expansion memory 774 can be provide as a security module for device 750, and can be programmed with instructions that permit secure use of device 750. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 764, expansion memory 774, or memory on processor 752 that can be received, for example, over transceiver 768 or external interface 762.

Device 750 can communicate wirelessly through communication interface 766, which can include digital signal processing circuitry where necessary. Communication interface 766 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through (radio-frequency) transceiver 768. In addition, short-range communication can occur, such as using a Bluetooth, Wi-Fi, or other such transceiver (not shown). In addition, GPS (Global Positioning System) receiver module 770 can provide additional navigation- and location-related wireless data to device 750, which can be used as appropriate by applications running on device 750.

Device 750 can also communicate audibly using audio codec 760, which can receive spoken information from a user and convert it to usable digital information. Audio codec 760 can likewise generate audible sound for a user, such as through a speaker, e.g., in a handset of device 750. Such sound can include sound from voice telephone calls, can include recorded sound, e.g., voice messages, music files, etc. and can also include sound generated by applications operating on device 750.

The computing device 750 can be implemented in a number of different forms, as shown in the figure. For example, it can be implemented as a cellular telephone 780. It can also be implemented as part of a smartphone 782, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A computer-implemented method for predicting a clinical outcome of a patient, the method comprising:
obtaining a pathology image associated with the patient;
processing the pathology image including:
determining a region of interest of the pathology image; and
segmenting the pathology image into a plurality of tiles, wherein each of the plurality of tiles has a uniform size;
providing the processed pathology image as an input to a machine learning model configured to annotate the pathology image;
obtaining, as an output of the machine learning model, the annotated pathology image associated with the patient, wherein the annotated pathology image includes annotations for different classes of tissues including a tumor regression; and
determining, based on the annotated pathology image, a metric indicative of the clinical outcome of the patient.

2. The computer-implemented method of claim 1, wherein the clinical outcome of the patient comprises responsiveness to one or more treatment regimens, survival, disease recurrence, and disease progression.

3. The computer-implemented method of claim 1, wherein the pathology image comprises a histopathology slide image.

4. The computer-implemented method of claim 1,
wherein the machine learning model has been trained on a plurality of training data items, and
wherein each training data item includes an annotated pathology image with tissue classes.

5. The computer-implemented method of claim 1, further comprising:
generating rendering data, when rendered by a user device, causes the user device to display a user interface that displays the annotated pathology image associated with the patient and the predicted clinical outcome of the patient.

6. The computer-implemented method of claim 5, wherein the user interface comprises a user selectable element to upload the pathology image for applying the machine learning model.

7. The computer-implemented method of claim 1, wherein the different classes of tissues comprise tumor, necrosis, area of tumor regression, and normal background, wherein the normal background indicates an uninvolved tissue.

8. (canceled)

9. (canceled)

10. The computer-implemented method of claim 1, wherein processing the pathology image further comprises:
normalizing color of the pathology image based on color of a set of pathology images; and
applying image transformations to the pathology image, wherein the image transformations include rotation, shift, flip, zoom, affine transformation, and adjusting brightness and contrast.

11. (canceled)

12. (canceled)

13. A system for predicting a clinical outcome of a patient comprising:
one or more computers; and
one or more storage devices storing instructions that are operable, when executed by the one or more computers, to cause the one or more computers to perform operations, the operations comprising:
obtaining a pathology image associated with the patient;
processing the pathology image including:
determining a region of interest of the pathology image; and
segmenting the pathology image into a plurality of tiles, wherein each of the plurality of tiles has a uniform size;
providing the processed pathology image as an input to a machine learning model configured to annotate the pathology image;
obtaining, as an output of the machine learning model, the annotated pathology image associated with the patient, wherein the annotated pathology image includes annotations for different classes of tissues including a tumor regression; and
determining, based on the annotated pathology image, a metric indicative of the clinical outcome of the patient.

14. The system of claim 13, wherein the clinical outcome of the patient comprises responsiveness to one or more treatment regimens, survival, disease recurrence, and disease progression.

15. The system of claim 13, wherein the pathology image comprises a histopathology slide image.

16. The system of claim 13,
wherein the machine learning model has been trained on a plurality of training data items, and
wherein each training data item includes an annotated pathology image with tissue classes.

17. The system of claim 13, the operations further comprising:
generating rendering data, when rendered by a user device, causes the user device to display a user interface that displays the annotated pathology image associated with the patient and the predicted clinical outcome of the patient.

18. The system of claim 17, wherein the user interface comprises a user selectable element to upload the pathology image for applying the machine learning model.

19. The system of claim 13, wherein the different classes of tissues comprise tumor, necrosis, area of tumor regression, and normal background, wherein the normal background indicates an uninvolved tissue.

20. The system of claim 13, wherein the metric indicative of the clinical outcome of the patient comprises a percent residual viable tumor, wherein the percent residual viable tumor is computed based on areas of the different classes of tissues in the pathology image.

21. The system of claim 13, wherein the metric indicative of the clinical outcome of the patient comprises a percent regression and a percent necrosis in the pathology image.

22. The system of claim 13, wherein processing the pathology image further comprises:
normalizing color of the pathology image based on color of a set of pathology images; and
applying image transformations to the pathology image, wherein the image transformations include rotation, shift, flip, zoom, affine transformation, and adjusting brightness and contrast.

23. One or more computer-readable storage medium storing instructions that, when executed by one or more computers, cause the one or more computers to perform operations for predicting a clinical outcome of a patient, the operations comprising:
obtaining a pathology image associated with the patient;
processing the pathology image including:
determining a region of interest of the pathology image; and
segmenting the pathology image into a plurality of tiles, wherein each of the plurality of tiles has a uniform size;
providing the processed pathology image as an input to a machine learning model configured to annotate the pathology image;
obtaining, as an output of the machine learning model, the annotated pathology image associated with the patient, wherein the annotated pathology image includes annotations for different classes of tissues including a tumor regression; and
determining, based on the annotated pathology image, a metric indicative of the clinical outcome of the patient.

24. The computer-readable storage medium of claim 13, wherein processing the pathology image further comprises:
normalizing color of the pathology image based on color of a set of pathology images; and
applying image transformations to the pathology image, wherein the image transformations include rotation, shift, flip, zoom, affine transformation, and adjusting brightness and contrast.
